(19) United States
(12) Patent Application Publication
(10) Pub. No.: US 2023/0158006 A1
(43) Pub. Date: May 25, 2023

(54) USE OF DIRECT THROMBIN INHIBITORS IN THE TREATMENT OF NEURODEGENERATIVE DISEASES

(71) Applicant: Paula Grammas, Lakewood Ranch, FL (US)

(72) Inventor: Paula Grammas, Lakewood Ranch, FL (US)

(21) Appl. No.: 17/919,264

(22) PCT Filed: Apr. 15, 2021
(86) PCT No.: PCT/US21/27574

§ 371 Date: Oct. 15, 2022

Related U.S. Application Data
(60) Provisional application No. 63/010,133, filed on Apr. 15, 2020.

(51) Int. Cl. A61K 31/4439; A61P 25/28

(52) U.S. Cl. CPC A61K 31/4439; A61P 25/28

(57) ABSTRACT

The present invention relates to the treatment of neurodegenerative diseases by administering an endothelial interrupter. More specifically, the endothelial interrupter is a direct thrombin inhibitor.

### CROSS REFERENCE TO RELATED APPLICATIONS

This application claims priority to and the benefit of U.S. Provisional Patent Application No. 63/010,133 filed on Apr. 15, 2020, the entire contents of which is incorporated by reference herein.

### STATEMENT REGARDING FEDERALLY SPONSORED RESEARCH OR DEVELOPMENT

This work was funded by a National Institutes for Health (NIH) grant and the United States government may have certain rights to this invention.

### FIELD OF THE INVENTION

The present invention relates to the treatment of neurodegenerative diseases by administering an endothelial interrupter. More specifically, the endothelial interrupter is a direct thrombin inhibitor.

### BACKGROUND

Neurodegenerative diseases, such as Alzheimer's Disease (AD), Parkinson's Disease (PD), and Huntington's Disease are characterized by the death of neurons within the human brain, leading to the deterioration of cognitive and motor function, and are a major contributor to dementia in the elderly, as well as decreased quality of life. There are about 5.7 million Americans diagnosed with AD.

Current attempts to treat neurodegenerative diseases have produced poor results, with the overwhelming majority of proposed treatments failing to pass clinical trials. 244 clinical trials for AD drugs were registered in clinicaltrials.gov from 2002 to 2012, but only one drug that is not an acetylcholinesterase inhibitor, the N-methyl-D-aspartate receptor antagonist memantine, received FDA-approval. Despite intense investigation, the exact causes of many neurodegenerative diseases remain poorly understood. Currently known pathologies, such as the deposition of amyloid R plaques in AD are not sufficient to fully explain the neuronal cell death that underlies AD dementia. Likewise, treatments that target amyloid R plaques and tangles of tau protein in neurons have failed to produce clinical results. The failure of disease-modifying drugs based on the AR cascade hypothesis for the past 30 years supports the idea that amyloid is but one factor in the complex pathogenesis of AD, and there is a need to identify new therapeutic targets to make progress in the AD field.

A more recent factor in AD pathogenesis is the role of vascular health within the brain. Specifically, micro-vessels isolated from AD brains overexpress a diverse array of neurotoxic and inflammatory proteins, which is consistent with the process of vascular activation, including thrombin, vascular endothelial growth factor, angiopoetin-2, tumor necrosis factor alpha (TN Fa), transforming growth factor beta, interleukin (IL) IL-18, IL-6, IL-8, monocyte chemoattractant protein-1 (MCP-1), nitric oxide, and matrix metalloproteinases (MMPs). Elevated vascular production of nitric oxide, a potentially neurotoxic mediator in the brain, may contribute to neuronal injury and death in AD. Pre-clinical studies using two AD animal models showed that a vascular activation inhibitor reduced vascular-derived neuroinflammation and improved cognitive performance. These data taken together suggest that vessels are dysfunctional in AD. The cerebral circulation as a target of injury in AD is likely because the brain endothelial cell is the only cell type in the CNS that is continuously exposed to potentially noxious elements and inflammatory mediators present in the blood.

One of the most important and yet overlooked aspects of the etiology and pathogenesis of neurodegenerative diseases is that they are typically age-related conditions. Models of aging brain circulation in rats show a change in smooth muscle reactivity and a significant increase in choline acetyltransferase in the cerebral arteries, as well as abnormalities in choline and peptide transport in the rat cerebral microcirculation. Age-related changes in cerebral micro-vessel membrane fluidity and protein and lipid composition have also been reported and are likely to be important for receptor/effector coupling and the efficiency of signal transduction cascades in the aged brain.

There is need in the art for more efficacious therapies for the treatment of neurodegenerative diseases, in particular, methods of treatments that incorporate deeper knowledge of the relationship between vascular activation and neurodegeneration.

### Definitions

In the following description, certain details are set forth such as specific quantities, sizes, etc. so as to provide a thorough understanding of the present embodiments disclosed herein. However, it will be obvious to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

Within the framework of the present description and in the subsequent claims, except where otherwise indicated, all numbers expressing amounts, quantities, percentages, and so forth, are to be understood as being preceded in all instances by the term “about”. As used herein, the term “about” is defined as ±5%. Also, all ranges of numerical entities include all the possible combinations of the maximum and minimum numerical values and all the possible intermediate ranges therein, in addition to those specifically indicated hereafter.

The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B and C as components.

The term “active form” herein refers to the metabolite form of the inactive prodrug that is metabolized within the body into its active form, regardless of the source of said prodrug.

The term “pharmaceutically acceptable salts or derivatives” herein refers to those salts or derivatives which possess the biological effectiveness and properties of the salified or derivatized compound and which do not produce adverse reactions when administered to a mammal, preferably a human. The pharmaceutically acceptable salts may be inorganic or organic salts; examples of pharmaceutically acceptable salts include but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemi sulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectianate, persulfate, phenylproprionate, picrate, pivalate, propionate, succinate, tartrate, thiocyanate, tosylate and undecanoate. Further information on pharmaceutically acceptable salts can be found in Handbook of pharmaceutical salts, P. Stahl, C. Wermuth, WILEY-VCH, 127-133, 2008, herein incorporated by reference. The pharmaceutically acceptable derivatives include the esters, the ethers and the N-oxides.

Salts derived from appropriate bases include alkali metal (e.g., sodium), alkaline earth metal (e.g., magnesium), ammonium and NW4+ (wherein W is C1-4 alkyl). Physiologically acceptable salts of a hydrogen atom or an amino group include salts or organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids. Physiologically acceptable salts of a compound with a hydroxy group include the anion of the compound in combination with a suitable cation such as Na+, NH4+, and NW4+ (wherein W is a C1-4 alkyl group).

Pharmaceutically acceptable salts include salts of organic carboxylic acids such as ascorbic, acetic, citric, lactic, tartaric, malic, maleic, isothionic, lactobionic, p-aminobenzoic and succinic acids; organic sulphonic acids such as methanesulphonic, ethanesulphonic, benzenesulphonic and p-toluenesulphonic acids and inorganic acids such as hydrochloric, sulphuric, phosphoric, sulphamic and pyrophosphoric acids.

For therapeutic use, salts of the compounds according to the invention will be pharmaceutically acceptable. However, salts of acids and bases that are not pharmaceutically acceptable may also find use, for example, in the preparation or purification of a pharmaceutically acceptable compound.

Preferred salts include salts formed from hydrochloric, sulfuric, acetic, succinic, citric and ascorbic acids.

As used herein, the term “chemically feasible” refers to a connectivity of atoms such that the chemical valency of each atom is satisfied. For example, an oxygen atom with two bonds and a carbon atom with four bonds are chemically feasible.

The term “physiologically acceptable excipient” herein refers to a substance devoid of any pharmacological effect of its own and which does not produce adverse reactions when administered to a mammal, preferably a human. Physiologically acceptable excipients are well known in the art and are disclosed, for instance in the Handbook of Pharmaceutical Excipients, sixth edition 2009, herein incorporated by reference.

As used herein, the term “neurodegenerative disease” means and refers to a disorder caused by the deterioration of certain nerve cells (neurons). Changes in these cells cause them to function abnormally, eventually bringing about their death or degeneration. Examples of such diseases include, but not limited to: Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of a-synuclein, diseases, disorders or conditions characterized by the presence of Lewy bodies, Creutzfeldt-Jakob, cerebral ischemias, epilepsy, neurodegenerative disease caused by traumatic injury, cognitive impairment, and combinations thereof.

The term “therapeutically effective amount” of a compound of the present invention refers to an amount which is effective in controlling or reducing the symptoms of a neurodegenerative disease. The term “controlling” is intended to refer to all processes wherein there may be a slowing, interrupting, arresting, or stopping of the progression of the disease and does not necessarily indicate a total elimination of all disease symptoms.

The term “therapeutically effective amount” is further meant to define an amount resulting in the improvement of any parameters or clinical symptoms characteristic of a neurodegenerative disease. The actual dose of the therapeutic agent will be different for the various specific molecules, and will vary with the patient's overall condition, the seriousness of the symptoms, and counterindications.

As used herein, the term “Alzheimer's Disease” (“AD”) means and refers to a progressive form of dementia that is similar to senile dementia. It is common that some of the first symptoms of AD are impaired memory which is followed by impaired thought and speech and can result in complete helplessness.

As used herein, the term “blocker”, “inhibitor”, “interrupter” or “antagonist” means a substance that retards or prevents a chemical or physiological reaction or response. Common blockers or inhibitors include but are not limited to antisense molecules, antibodies, antagonists and their derivatives.

As used herein, the term “patient” means and refers to a human or animal.

As used herein, the term “dabigatran” or “dabigatran etexilate”, sold under the brand name Pradaxa® among others, is an anticoagulant used to the treat and prevent blood clots and to prevent stroke in people with atrial fibrillation. It has a formula of C34H41N7O5, a molar mass of 627.734 g/mol, and the following structure:

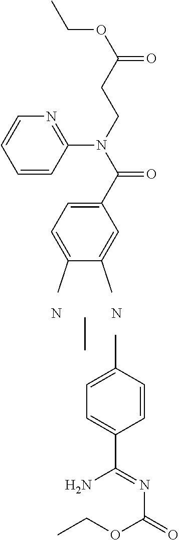

### SUMMARY

The invention is considered to be a method of treatment of a neurodegenerative disease by the administration of an effective amount of a therapeutic agent which inhibits vascular activation. In a preferred embodiment, the therapeutic agent is a direct thrombin inhibitor. In a preferred embodiment, the direct thrombin inhibitor is dabigatran.

Factors and processes characteristic of vascular activation and angiogenesis have been documented in the AD brain. Expression of these factors is consistent with the process of vascular activation and reflects the transition of endothelial cells from a quiescent to a highly synthetic phenotype. These vascular-derived factors are directly injurious to neurons but can also cause injury indirectly by activating neighboring glial cells to release reactive oxygen species and inflammatory factors. If vascular-derived factors contribute to a cascade of events that lead to dementia in the AD brain then blocking or decreasing vascular activation, and the subsequent release of neurotoxic factors, could improve cognitive performance. We propose targeting vascular activation as a new approach to developing disease-modifying therapies for AD.

Thrombin, an inflammatory protein upregulated in AD, is a key mediator of cerebrovascular inflammation in AD and diminishes vascular activation in AD models. Thrombin also blocks hypoxia-induced cerebrovascular inflammation in vitro and also induces NADPH-mediated oxidative stress. When administered intravenously, thrombin evokes deficits in reference memory, increases task latency, increases in TUNEL+ cells, and stimulates astrogliosis.

In AD models, the thrombin inhibitor dabigatran mitigates the neurotoxicity of thrombin by reducing the production of TNFa, IL-6, IL-8, MMPs, and other inflammatory proteins from endothelial cells. In non-published results from our laboratory with transgenic Tg4510 AD mice, short treatment with dabigatran was responsible for lowering brains' oxidative stress.

This novel idea is consistent with studies linking cardiovascular risk factors to vascular dysfunction and neuroinflammation as previously described. Dabigatran is marketed as an anticoagulant for the prevention of strokes in people with atrial fibrillation. The use of dabigatran in the treatment of a neurodegenerative disease is novel and not predicted by the prior art on the drug.

Various further embodiments disclose methods for reducing a patient's endothelial cell release of at least one inflammatory cytokine, the methods comprising the step of administering a medicament comprising a therapeutic agent which inhibits vascular activation. In various embodiments, the inflammatory proteins are chosen from the group consisting of TN Fa, IL-6, IL-8, or MMP2. In a further embodiment, the level of oxidative stress in the patient's brain is reduced.

Various further embodiments disclose a method of improving the cognitive function of a patient suffering from a neurodegenerative disease, the method comprising the step of administering a therapeutically effective amount of a therapeutic agent which inhibits vascular activation.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

FIG. 1 shows levels of mRNA expression of HIF-1a, thrombin, IL-6, MCP-1, and MMP2 after 6 hours hypoxia as determined by real time PCR in homogenized brain tissues from control (C), control+dabigatran (DTI), AD, and AD+DTI mice. Data are from four mice per group and expressed as fold change over control (untreated). ***p<0.001 vs. C (control); ap<0.001, bp<0.01 vs. H (hypoxia).

FIG. 2 shows brain tissues from control (C), control+dabigatran (DTI), AD, and AD+DTI mice immunostained with HIF-1a, thrombin, IL-6, MCP-1, and MMP2 primary antibodies and fluorescence labeled secondary antibody (green). The bar graph denotes signal intensities normalized to endothelial specific marker von Willebrand factor (vWF, red) and control values set to 1. Data are from four mice per group. *p<0.05, ***p<0.001 vs. C (control); ap<0.001, cp<0.05 vs. AD.

FIG. 3 shows dabigatran reduces expression of HIF-1a, thrombin, IL-6, MCP-1, AND MMP2 in the brains of AD transgenic mice. Brain tissues from control, control+DTI, AD and AD+DTI mice were homogenized, total RNA collected, reverse transcribed and mRNA expression of HIF-1a, thrombin, IL-6, MCP-1, and MMP2 determined by real time PCR. Data are from four mice per group and expressed as fold change over control (C, untreated). *** p<0.001 vs. C (control); ap<0.001, bp<0.01 vs. AD.

FIG. 4 shows generation of ROS in AD mice and hypoxic endothelial cell cultures is inhibited by dabigatran. Brain tissue sections from frontal cortices from control, control+DTI, AD, and AD+DTI mice were incubated for 30 min with 5 pM of Dihydroethidium (DHE, red) fluorescence dye, and NucBlue stain (blue). Data represents signal intensities of DHE stained cells to non-stained cells. ***p<0.001 vs. control (C); ap<0.001 vs. AD.

FIG. 5 is a bar graph reporting the effect of Dabigatran on the thrombin levels of Tg4510 mice after one week of treatment. The Y-axis is the fold change over control. The left bar represents the Wild-type control mice, the middle bar represents the Tg-Vehicle and the right bar represents the Tg-Dabigatran treated mice.

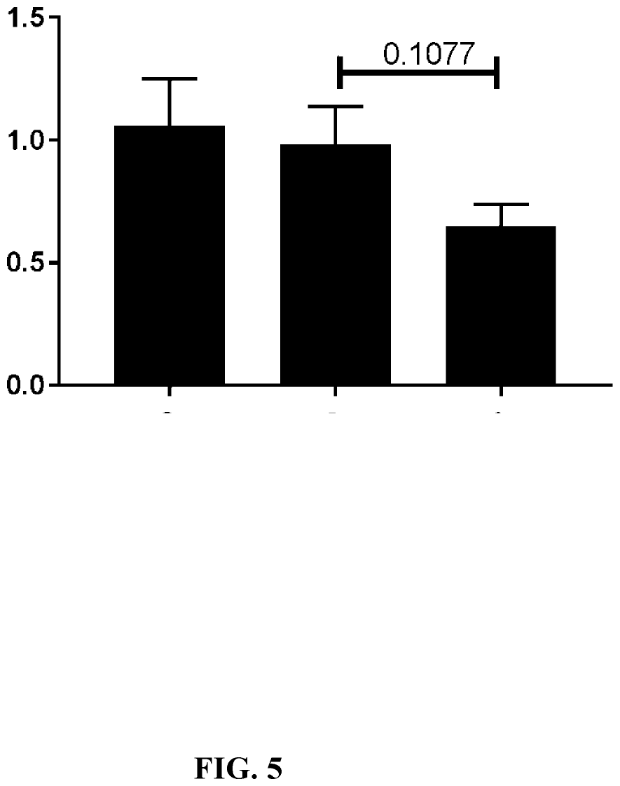

FIG. 6 is a bar graph reporting the effect of Dabigatran on the prothrombin levels of Tg4510 mice after one week of treatment. The Y-axis is the fold change over control. The left bar represents the Wild-type control mice, the middle bar represents the Tg-Vehicle and the right bar represents the Tg-Dabigatran treated mice.

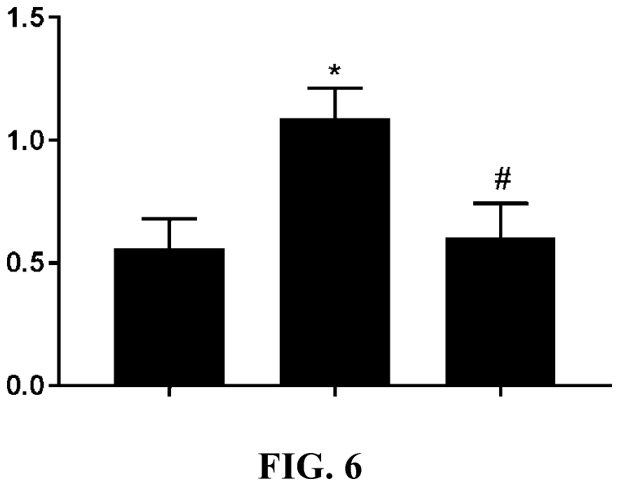

FIG. 7 is a bar graph reporting the effect of Dabigatran on the fibrin levels of Tg4510 mice after one week of treatment. The Y-axis is the fold change over control. The left bar represents the Wild-type control mice, the middle bar represents the Tg-Vehicle and the right bar represents the Tg-Dabigatran treated mice.

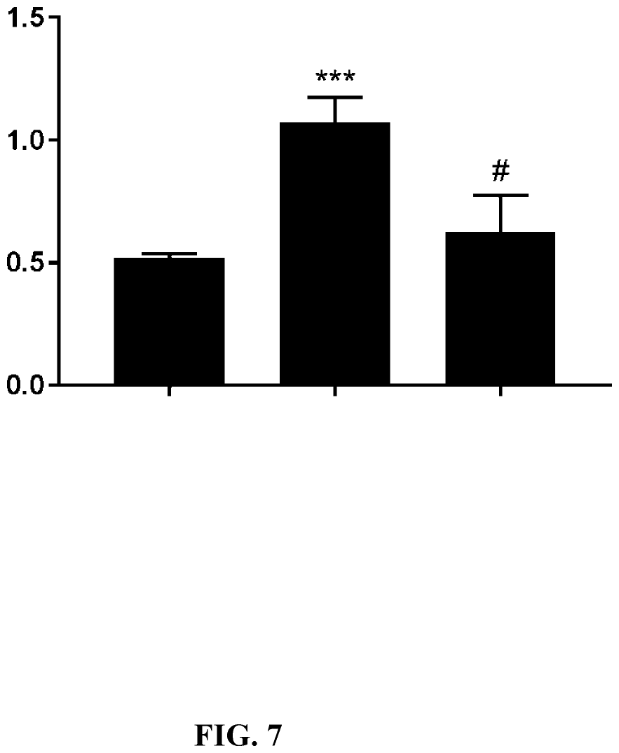

FIG. 8 is a bar graph reporting the effect of Dabigatran on the NOX4 marker of oxidative stress of Tg4510 mice after one week of treatment. The Y-axis is the fold change over control. The left bar represents the Wild-type control mice, the middle bar represents the Tg-Vehicle and the right bar represents the Tg-Dabigatran treated mice.

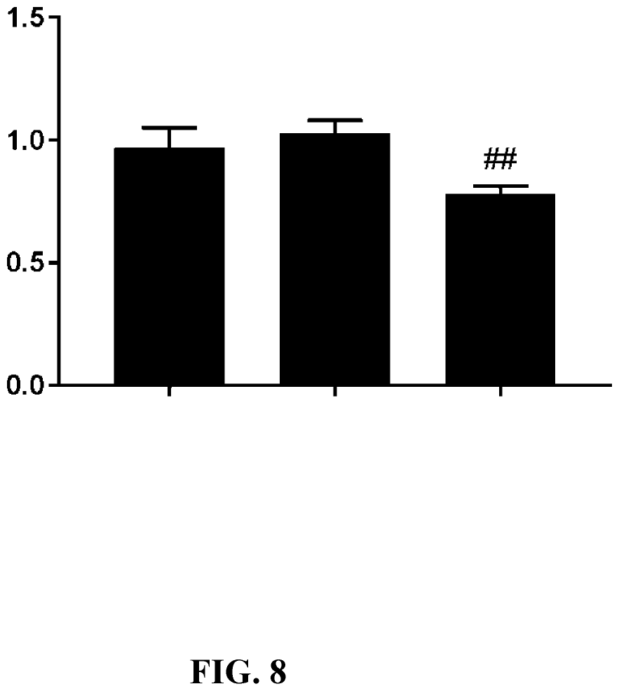

FIG. 9 is a bar graph reporting the effect of Dabigatran on the iNOS marker of oxidative stress of Tg4510 mice after one week of treatment. The Y-axis is the fold change over control. The left bar represents the Wild-type control mice, the middle bar represents the Tg-Vehicle and the right bar represents the Tg-Dabigatran treated mice.

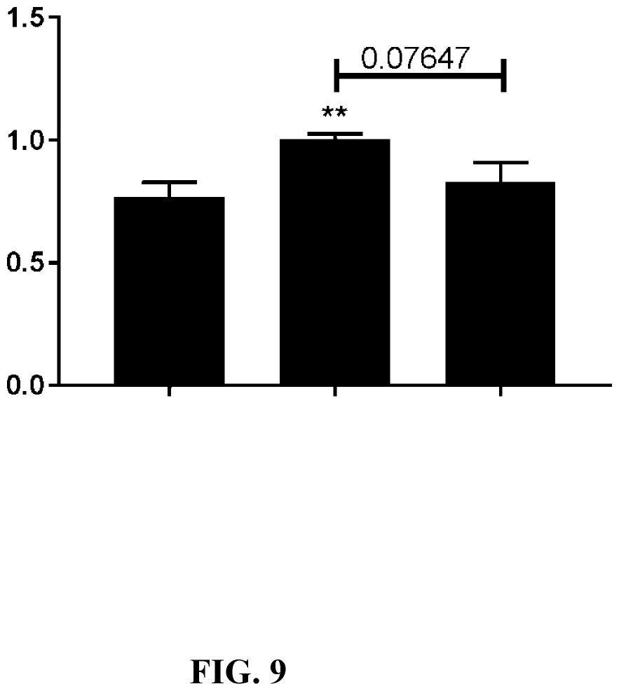

FIG. 10 is a bar graph reporting the effect of Dabigatran on the expression of anti-oxidative stress protein SOD1 of Tg4510 mice after one week of treatment. The Y-axis is the fold change over control. The left bar represents the Wild-type control mice, the middle bar represents the Tg-Vehicle and the right bar represents the Tg-Dabigatran treated mice.

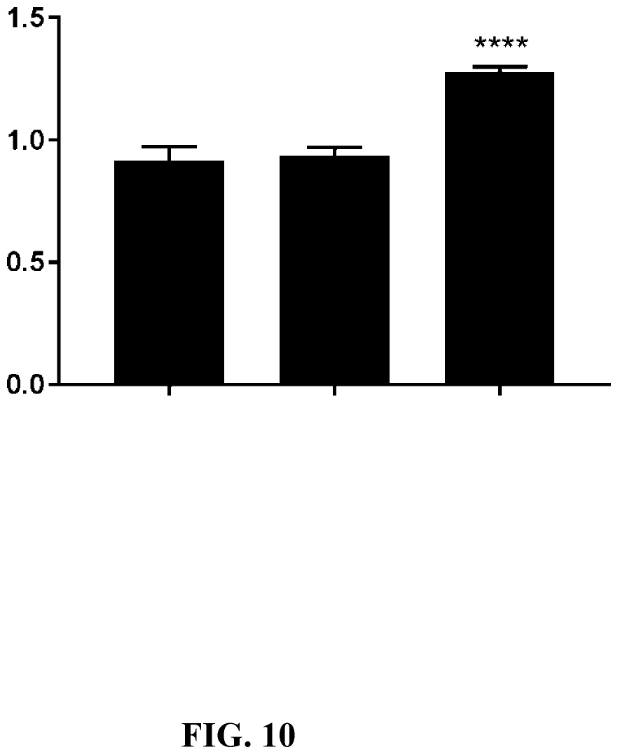

FIG. 11 is a bar graph reporting the effect of Dabigatran on the expression of anti-oxidative stress protein SOD2 of Tg4510 mice after one week of treatment. The Y-axis is the fold change over control. The left bar represents the Wild-type control mice, the middle bar represents the Tg-Vehicle and the right bar represents the Tg-Dabigatran treated mice.

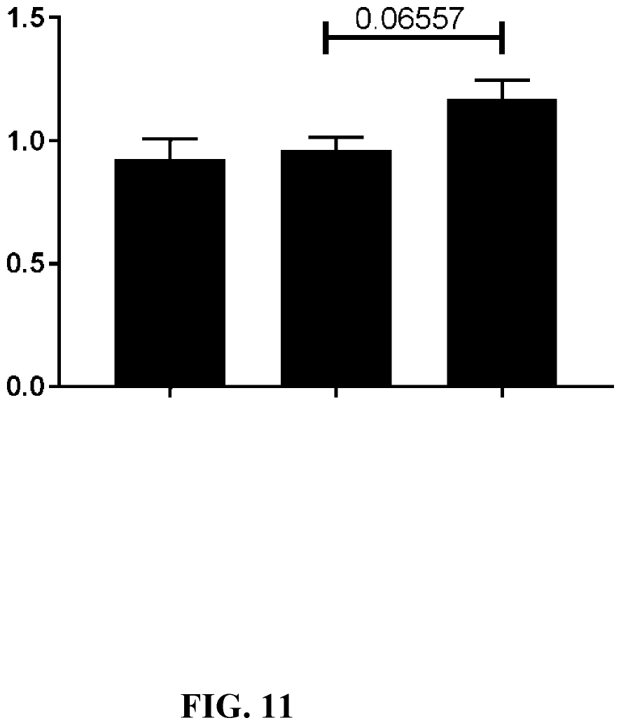

FIG. 12 is a bar graph reporting the effect of Dabigatran on the GFAP marker of neuroinflammation of Tg4510 mice after one week of treatment. The Y-axis is the fold change over control. The left bar represents the Wild-type control mice, the middle bar represents the Tg-Vehicle and the right bar represents the Tg-Dabigatran treated mice.

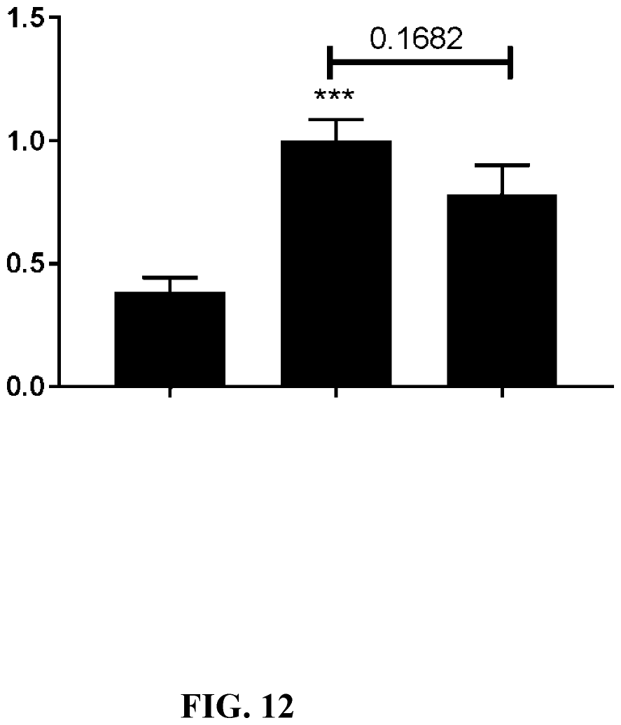

FIG. 13 is a bar graph reporting the effect of Dabigatran on the Ibal marker of neuroinflammation of Tg4510 mice after one week of treatment. The Y-axis is the fold change over control. The left bar represents the Wild-type control mice, the middle bar represents the Tg-Vehicle and the right bar represents the Tg-Dabigatran treated mice.

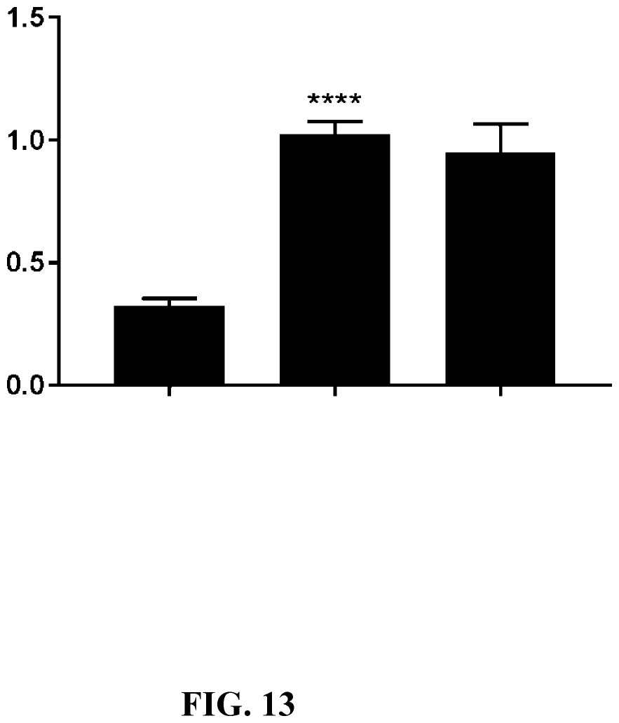

FIG. 14A is a bar graph reporting the effect of Dabigatran on the thrombin expression of 21-month-old APP/PS1 mice after one week of treatment. The Y-axis is expression normalized to B-actin. The left bar represents the DMSO control and the right bar represents the Dabigatran treated mice. FIG. 14B is a bar graph reporting the effect of Dabigatran on the fibrin expression of 21-month-old APP/PS1 mice after one week of treatment. The Y-axis is expression normalized to B-actin. The left bar represents the control and the right bar represents the Dabigatran treated mice.

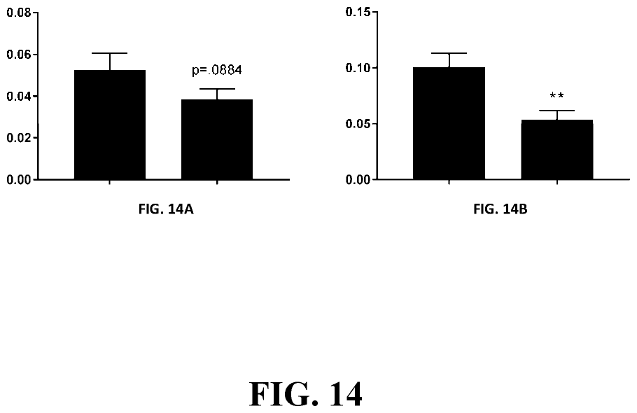

FIG. 15A is a bar graph reporting the effect of Dabigatran on the expression of oxidative stress protein iNOS of 21-month-old APP/PS1 mice after one week of treatment. The Y-axis is expression normalized to B-actin. The left bar represents the DMSO control and the right bar represents the Dabigatran treated mice. FIG. 15B is a bar graph reporting the effect of Dabigatran on the expression of oxidative stress protein eNOS of 21-month-old APP/PS1 mice after one week of treatment. The Y-axis is expression normalized to B-actin. The left bar represents the DMSO control and the right bar represents the Dabigatran treated mice. FIG. 15C is a bar graph reporting the effect of Dabigatran on the expression of oxidative stress protein NOX4 of 21-month-old APP/PS1 mice after one week of treatment. The Y-axis is expression normalized to B-actin. The left bar represents the DMSO control and the right bar represents the Dabigatran treated mice.

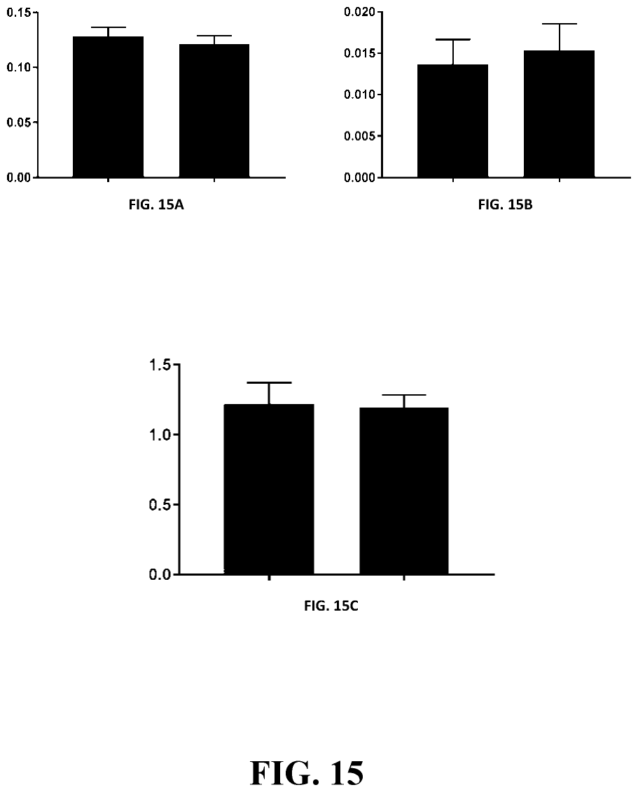

FIG. 16 is a bar graph reporting the effect of Dabigatran on the expression of GFAP 21-month-old APP/PS1 mice after one week of treatment. The Y-axis is expression normalized to B-actin. The left bar represents the DMSO control and the right bar represents the Dabigatran treated mice.

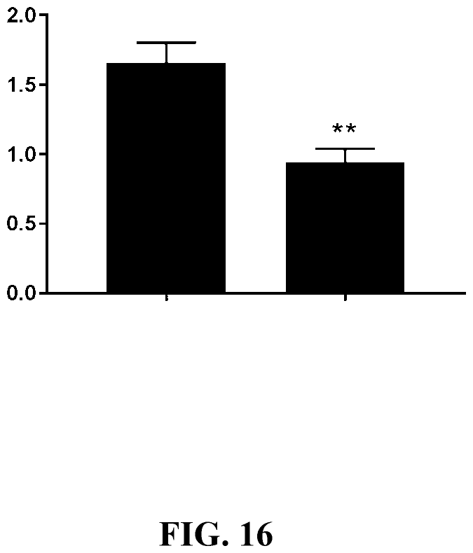

FIG. 17 reports the effect of thrombin and ApoE isoforms on TNFα. In particular, Western blot expressions of TNFα in human astrocytes treated in vitro with ApoE isoforms (20 nM) with or without thrombin (50 nM) for twenty-four (24) hours are provided. FIG. 17A is the Western blot expression for intracellular TNFα whereas FIG. 17B is the Western blot expression for secreted TNFα.

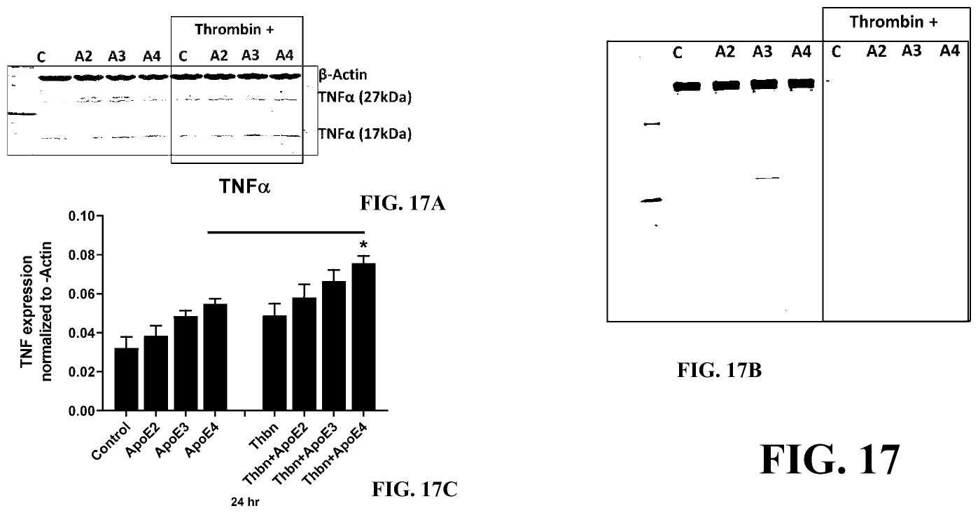

FIG. 18 reports the effect of thrombin and ApoE isoforms on IL1β (FIG. 18A) and IL-6 (FIG. 18B).

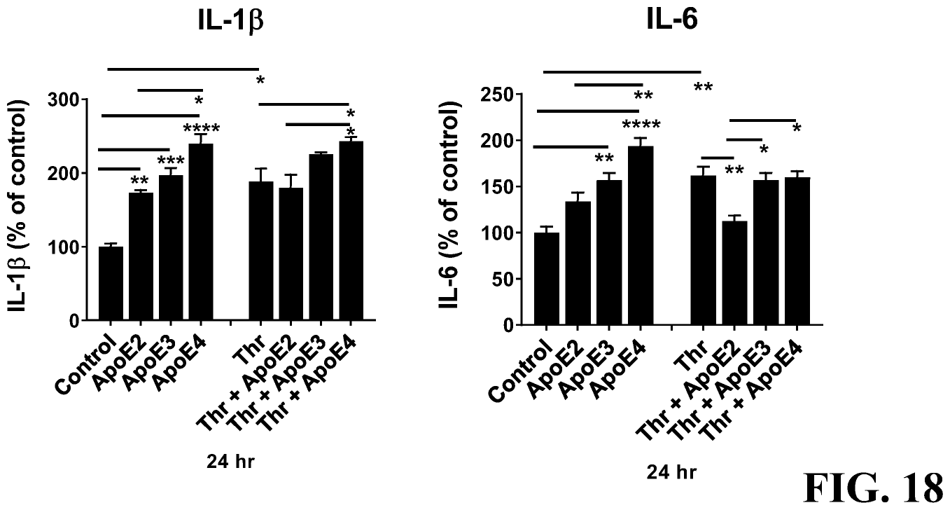

FIG. 19 reports the effect of thrombin and ApoE isoforms on secreted BDNF. FIG. 19A is a Western blot expression of secreted BDNF in conditioned media from human astrocytes treated in vitro with ApoE isoforms (20 nM) with and without thrombin (50 nM) for twenty-four (24) hours.

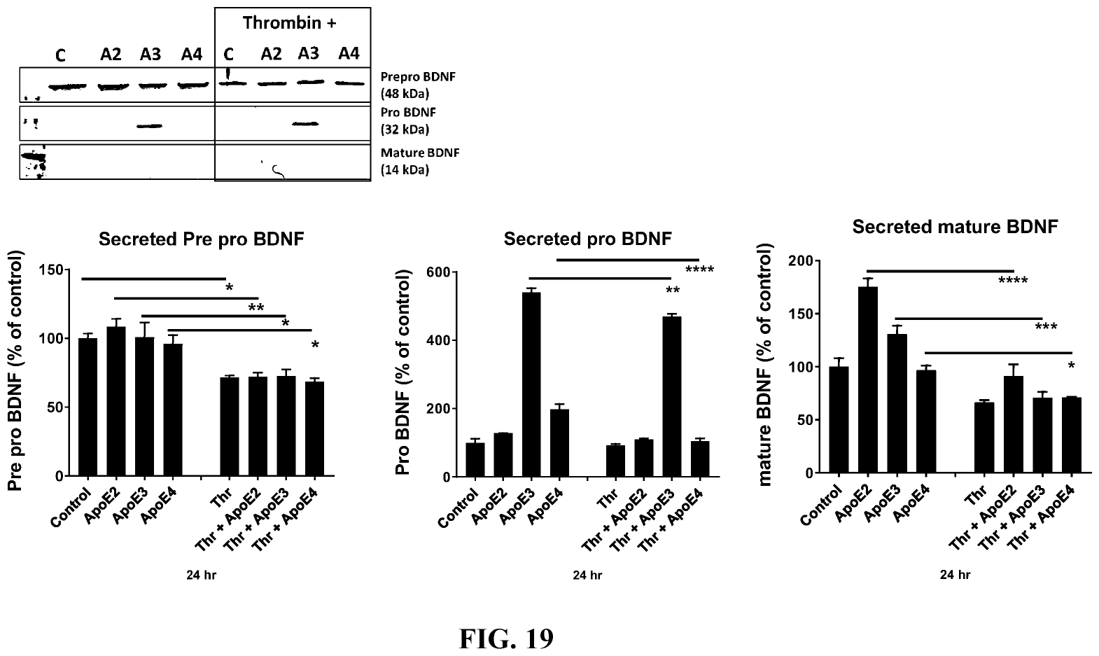

FIG. 20 reports the effect of thrombin and ApoE isoforms on NOX4 and iNOS expression. FIG. 20A is a Western blot expression of iNOS and NOX4 in human astrocytes treated in vitro with ApoE isoforms (20 nM) with and without thrombin (50 nM) for twenty-four (24) hours.

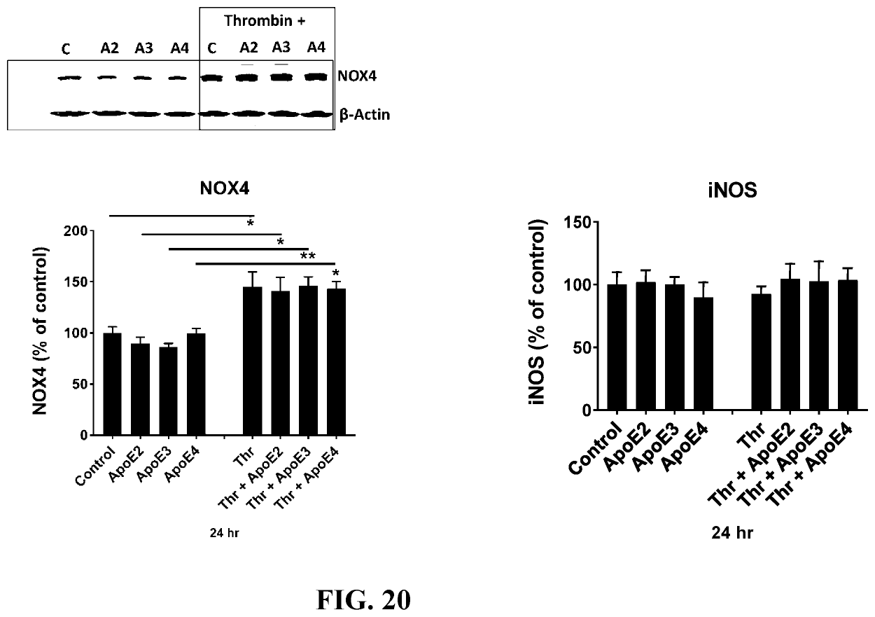

FIG. 21 reports the effect of thrombin on primary human astrocytes. Human astrocytes were grown in vitro and thereafter treated with three (3) different doses of thrombin (10 nM, 50 nM and 100 nM, respectively) for twenty-four (24) hours. Western blot expressions for BDNF (Pro and mature) and IL-8, as well as bar graphs depicting the observed levels of expression, are provided.

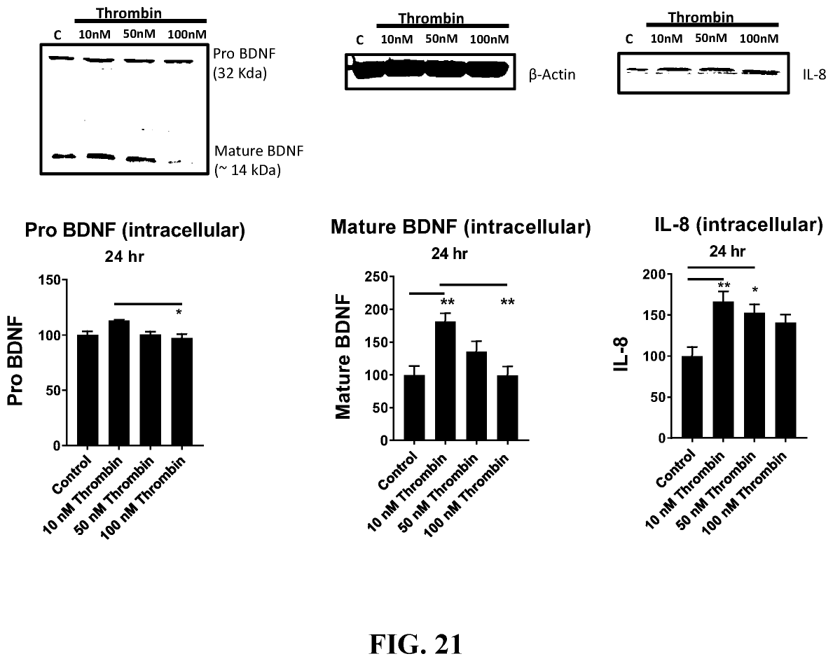

### DETAILED DESCRIPTION OF THE INVENTION

We hypothesize that abnormal, pathologically altered brain endothelium produces factors that are toxic to neurons. Therefore, the cerebral vasculature could be an important mediator of neuronal injury in neurodegenerative diseases.

In AD, thrombin has been detected in the senile plaques characteristic of this disease. Traumatic brain injury where neurons are exposed to high thrombin levels is associated with an increased incidence of AD. Some neurologic diseases, such as AD and Parkinson's disease are characterized by increased levels of both thrombin and the thrombin receptor protease-activated receptor 1 (PAR-1). Furthermore, immunoreactivity for the major brain thrombin inhibitor, protease nexin-1 is found to be significantly decreased in AD brains, particularly around blood vessels, suggesting vascular release of thrombin. Our laboratory has shown, by RT-PCR that brain blood vessels isolated from AD patients, but not age-matched controls, synthesize thrombin.

The multifunctional protease thrombin causes neuronal cell death both in vitro and in vivo. Thrombin causes rapid tau aggregation. Intracerebroventricular administration of thrombin directly into the rat brain results in neuronal cell death, glial scarring and cognitive deficits. Activation or over-expression of the receptor PAR-1 has been shown to induce motor neurodegeneration. Thrombin exerts direct neurotoxicity by several mechanisms including reentry into the cell cycle, induction of pro-apoptotic proteins, as well as via NADPH-oxidase mediated oxidative stress.

Thrombin causes endothelial activation and enhanced expression and/or release of many proinflammatory proteins including MCP-1 and ICAM-1, both of which are upregulated in the cerebrovasculature in AD. The cellular action of thrombin, a potent angiogenic factor, on endothelial cells may represent an important early event in vascular activation. Blocking thrombin reduces the activation of brain-derived endothelial cells to hypoxia.

Inhibiting thrombin decreases cerebrovascular inflammation/activation and reactive oxygen species generation in AD mice. Immunofluorescent analysis of the cerebrovasculature in AD mice demonstrates significant increases in thrombin, hypoxia inducible factor-1a (HIF-1a), IL-6, MCP-1, MMPs, and reactive oxygen species compared to controls. Administration of the thrombin inhibitor dabigatran (100 mg/kg) to AD mice for 34 weeks significantly decreases expression of these of inflammatory proteins and reactive oxygen species.

In summary, thrombin is elevated in the brain and cerebral microvasculature in AD and is neurotoxic. Thrombin inhibitors block the proinflammatory effects of hypoxia on brain endothelial cells and reduce vascular activation in transgenic AD mice. Based on data that reducing cerebrovascular activation is associated with improved cognition, thrombin inhibitors could prove useful for improving cognition in AD patients.

A therapeutically effective amount of the compound used in the treatment described herein can be readily determined by the attending diagnostician, as one skilled in the art, by the use of conventional techniques and by observing results obtained under analogous circumstances. In determining the therapeutically effective dose, a number of factors are considered by the attending diagnostician, including, but not limited to: the weight, age, and general health of the subject; the degree of or the severity of the disease; the response of the individual patient; the particular compound administered; the mode of administration; the bioavailability characteristic of the preparation administered; the dose regimen selected; the use of concomitant medication; and other relevant circumstances.

Preferred amounts and modes of administration are able to be determined by one skilled in the art. One skilled in the art of preparing formulations can readily select the proper form and mode of administration depending upon the particular characteristics of the compound selected the disease state to be treated, the stage of the disease, and other relevant circumstances using formulation technology known in the art, described for example in Remington's Pharmaceutical Sciences, latest edition, Mack Publishing Co.

Pharmaceutical compositions can be manufactured utilizing techniques known in the art. Typically the therapeutically effective amount of the compound will be admixed with a pharmaceutically acceptable carrier.

Therapeutic agents contemplated herein may be administered by a variety of routes, for example, orally or parenterally (i.e., subcutaneously, intravenously, intramuscularly, intraperitoneally, or intratracheally).

The pharmaceutical compositions of this invention may be orally administered in any orally acceptable dosage form including, but not limited to, capsules, tablets, aqueous suspensions or solutions. In the case of tablets for oral use, carriers that are commonly used include lactose, sucrose, and corn starch in combination with binders, such as acacia, cornstarch, or gelatin, disintegrating agents such as potato starch or alginic acid, and a lubricant such as stearic acid or magnesium stearate. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

In various embodiments, the compounds of the invention may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. Further pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The pharmaceutical carrier may also contain preservatives, and buffers as are known in the art.

For surgical implantation, the active ingredients may be combined with any of the well-known biodegradable and bioerodible carriers, such as polylactic acid, hyaluronic acid and collagen formulations. Such materials may be in the form of solid implants, sutures, sponges, wound dressings, and the like. In any event, for local use of the materials, the active ingredients usually are present in the carrier or excipient in a weight ratio of from about 1:1000 to 1:20,000, but are not limited to ratios within this range. Preparation of compositions for local use are detailed in Remington's Pharmaceutical Sciences, latest edition, (Mack Publishing).

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be affected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of inhibitor that may be combined with the carrier materials to produce a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of inhibitor will also depend upon the particular compound in the composition.

Depending upon the particular neurodegenerative disease condition to be treated or prevented, additional drugs, which are normally administered to treat or prevent that condition may be administered together with the inhibitors of this invention. For example, chemotherapeutic agents or other anti-proliferative agents may be combined with the endothelial interrupters of this invention to treat proliferative diseases.

Those additional agents may be administered separately, as part of a multiple dosage regimen, from the endothelial interrupter-containing composition. Alternatively, those agents may be part of a single dosage form, mixed together with the endothelial interrupter in a single composition.

Additional pharmaceutical methods may be employed to control the duration of action. Controlled release preparations may be achieved through the use of polymers to complex or absorb the active ingredient. The controlled delivery may be achieved by selecting appropriate macromolecules (for example, polyesters, polyamino acids, polyvinyl, pyrrolidone, ethylenevinylacetate, methylcellulose, carboxymethylcellulose, or protamine, sulfate) and the appropriate concentration of macromolecules as well as the methods of incorporation, in order to control release.

Another possible method useful in controlling the duration of action by controlled release preparations is incorporation of the active agent into particles of a polymeric material such as polyesters, polyamino acids, polysaccharides, hydrogels, poly(lactic acid), or ethylene vinylacetate copolymers.

Alternatively, instead of incorporating the active agent into polymeric particles, it is possible to entrap these materials in microcapsules prepared, for example, by coacervation techniques or by interfacial polymerization (for example, hydroxymethylcellulose or gelatine-microcapsules and poly(methylmethacylate) microcapsules, respectively), in colloidal drug delivery systems (for example, liposomes, albumin microspheres, microemulsions, nano-particles, and nanocapsules), or in macroemulsions. Such techniques are disclosed in the latest edition of Remington's Pharmaceutical Sciences.

U.S. Pat. No. 4,789,734 describe methods for encapsulating biological materials in liposomes. Essentially, the material is dissolved in an aqueous solution, the appropriate phospholipids and lipids added, along with surfactants if required, and the material dialyzed or sonicated, as necessary. A good review of known methods is by G. Gregoriadis, Chapter 14. “Liposomes”, Drug Carriers in Biology and Medicine, pp. 287-341 (Academic Press, 1979). Microspheres formed of polymers or proteins are well known to those skilled in the art and can be tailored for passage through the gastrointestinal tract directly into the blood stream. Alternatively, the agents can be incorporated and the microspheres, or composite of microspheres, implanted for slow release over a period of time, ranging from days to months. See, for example, U.S. Pat. Nos. 4,906,474, 4,925,673, and 3,625,214.

When the composition is to be used as an injectable material, it can be formulated into a conventional injectable carrier. Suitable carriers include biocompatible and pharmaceutically acceptable phosphate buffered saline solutions, which are preferably isotonic. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

For reconstitution of a lyophilized product in accordance with this invention, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field.

The pharmaceutical composition may also be in the form of an aqueous solution containing many of the same substances as described above for the reconstitution of a lyophilized product.

The compounds can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Examples of suitable acid salts include acetate, adipate, alginate, aspartate, benzoate, benzenesulfonate, bisulfate, butyrate, citrate, camphorate, cam phorsulfonate, cyclopentanepropionate, digluconate, dodecyl sulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium and potassium), alkaline earth metal (e.g., magnesium), ammonium and N—(C1-4 alkyl)4+salts. This invention also envisions the quaternization of any basic nitrogen-containing groups of the compounds disclosed herein. Water or oil-soluble or dispersible products may be obtained by such quaternization.

As mentioned above, the products of the invention may be incorporated into pharmaceutical preparations which may be used for therapeutic purposes. However, the term “pharmaceutical preparation” is intended in a broader sense herein to include preparations containing a protein composition in accordance with this invention, used not only for therapeutic purposes but also for reagent or diagnostic purposes as known in the art, or for tissue culture. The pharmaceutical preparation intended for therapeutic use should contain a “pharmaceutically acceptable” or “therapeutically effective amount” of a vascular activation inhibitor or antibody, i.e., that amount necessary for preventative or curative health measures. If the pharmaceutical preparation is to be employed as a reagent or diagnostic, then it should contain reagent or diagnostic amounts of a vascular activation inhibitor or antibody.

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes to the claims that come within the meaning and range of equivalency of the claims are to be embraced within their scope. Further, all published documents, patents, and applications mentioned herein are hereby incorporated by reference, as if presented in their entirety.

### EXEMPLIFICATION

### Methods of Experimentation:

Culture and hypoxic exposure of rat brain endothelial cells: Brain endothelial cell cultures were obtained from rat brain micro-vessels, as previously described (Diglio et al., 1993). The purity of these cultures was confirmed using antibodies to the endothelial cell surface antigen Factor VIII. Endothelial cells used in this study (passages 8-15) were maintained in Dulbecco's modified Eagle's medium (DMEM, Sigma-Aldrich, St. Louis, Mo.) supplemented with 10% fetal bovine serum (FBS), 1% antibiotic/antimycotic, and 2 mM glutamine. Confluent endothelial cell cultures were washed three times with Hank's balanced salt solution (HBSS, Gibco, Grand Island, N.Y.) and then incubated at 37° C. with serum-free DMEM for 6 h under hypoxic (1% 02) or normoxic (21% 02) conditions.

Measurement of cell survival by MTT assay: Cells were washed with phosphate buffer saline (PBS) and incubated with the MTT reagent 3-(4,5-dimethylthiazol-2-yl)-2-5-diphenyl tetrazolium bromide (1:40 dilution) for 5-10 min at 37° C. The cells convert the MTT reagent to formazan which is quantified by colorimetric assay (Cell Titer 95 Aqueous solution cell proliferation assay, Promega, Madison, Wis.). The formazan product was read at 490 nm. The number of control cells, i.e., viable cells not exposed to any treatment, was defined as 100%.

Administration of thrombin inhibitor to mice and immunofluorescent staining of mice brain sections: Adult wild-type 3×TgAD—LaFerla (control) and 3×TgAD—LaFerla mice were purchased at 8 weeks of age from The Jackson Laboratory (Bar Harbor, Me.). Daily administration of the orally available direct thrombin inhibitor (DTI) dabigatran etexylate (Pradaxa®, Boehringer Ingelheim, Germany) (100 mg/kg) in PBS to AD mice and vehicle to control mice began at 18 weeks of age and continued daily for 34 weeks. DTI was administered in food. To ensure all food, and therefore drug, was consumed while maintaining ad libitum weight, mice were weighed, and food intake monitored daily. All animal procedures were performed in accordance with NIH “Guide for the Care and Use of Laboratory Animals” and Texas Tech University Health Sciences Center Institutional Animal Care and Use Committee (IACUC) guidelines.

Mice were euthanized, and brain tissue fixed with 10% neutral buffered formalin (NBF). The brain was removed and 1 mm blocks of tissue from cerebral cortex were post fixed in 10% NBF for additional 12 h and embedded in paraffin. Brain sections from the frontal cortex (7 pm thick) were deparaffinized in xylene, hydrated through a graded alcohol series, and then rinsed for 5 min in deionized water. Sections were subjected to heat-induced epitope antigen retrieval, washed with Tris-buffered saline with Tween (TBST) and blocked with 10% donkey serum at room temperature for 2 h. The sections were incubated at 4° C. overnight with primary antibodies against HIF-1 a (abl, Abcam, Cambridge, Mass.), thrombin (sc16972, Santa Cruz Biotechnology, Santa Cruz, Calif.), MMP2 (ab37150, Abcam), IL-6 (ab6672, Abcam), MCP-1 (ab9858, Abcam), or the endothelial cell marker von Willebrand Factor (vWF, sc114014, sc8068, Santa Cruz) in TBS containing 2.5% donkey serum. Sections were then washed, blocked, and incubated with appropriate secondary antibodies conjugated with Alexa Fluor 488 or Alexa Fluor 594. Sections (3 per mouse) were incubated with DAPI solution at room temperature for 25 min and viewed using an Olympus IX71 microscope and quantified with HAMAMATSU imaging software.

Detection of reactive oxygen species in cell culture and mouse brain tissue sections: Brain endothelial cell cultures were grown on coverslips in 24-well plates, incubated at 37° C. in serum free media with or without dabigatran (DTI, 1 nM) and exposed to hypoxic (6 h, 1% 02) or normoxic (21% 02) conditions. Sections from frozen brain (10-20 pm thickness) were placed on coverslips in 24-well plates with 0.5 ml of PBS at room temperature. Brain sections and cell cultures were incubated with 5 pM of dihydroethidium (DH E) fluorescence dye (Life Technologies, Grand Island, N.Y.; D23107) and Hoechst33342 NucBlue® stain (Life Technologies, R37605) for 30 min in the dark at room temperature. DHE is a cell permeable compound which reacts with intracellular ROS to form oxyethidium, which emits a bright red color detectable by fluorescent microscopy. Following two washes with PBS, images were captured immediately using an Olympus IX71 microscope and analyzed using HAMAMATSU imaging software.

Real-time PCR analysis: RNA from cultured rat brain endothelial cells was prepared using the TRI Reagent RT (Molecular Research Center, Inc., Cincinnati, Ohio), according to manufacturer protocol. Four micrograms of total RNA were reverse transcribed using oligo dT primers and Transcriptor high fidelity reverse transcriptase according to the manufactures protocol (Roche Applied Science). Real-time PCR was performed using the Applied Biosystems 7900 HT fast real time PCR system. Taqman gene expression master mix and Taqman gene expression assays from Applied Biosystems (Weiterstadt, Germany) were used for PCR. Primers for Thrombin (rat: Assay ID Rn00575908_m1, mice: Mm00438843_m1), HIF-1 a (rat: Assay ID Rn00577560_m1, mice: Mm00468869_m1), MMP2 (rat: Assay ID Rn01538170_m1, mice: Mm00439498_m1), IL-6 (rat: Assay ID Rn01410330_m1, mice: Mm00446190_m1), MCP-1 (rat: Assay ID Rn00580555_m1, mice: Mm00441242_m1), and actin (rat: Assay ID Rn00667869_m1, mice: Mm00607939_s1) were used for these experiments. Results were normalized to actin. Fold difference between two samples (relative quantification) was determined by use of the delta-delta method [S1/S2=2−(T1−T2)], where S1 and S2 represent samples 1 and 2, and T1 and T2 represent the threshold cycles of samples 1 and 2, respectively.

Statistical analysis: Data from each experiment are expressed as mean±standard deviation (SD). The One-Way ANOVA followed by Bonferroni's comparison tests were performed for multiple samples. Statistical significance was determined at p<0.05.

### Experiment 1: Thrombin Inhibition Affects the Inflammatory Response of Brain Endothelial Cells Exposed to Hypoxia

Brain micro-vessel endothelial cell cultures were incubated with the DTI dabigatran (10 pM-100 nM), exposed to hypoxia for 6 h and cell viability measured by MTT assay. There was a modest (19%) but significant (p<0.05) increase in cell survival at 1-10 nM DTI while DTI doses in excess of 100 nM were toxic. Exposure of endothelial cultures to DTI under normoxic conditions did not affect cell survival at doses under 100 nM (data not shown).

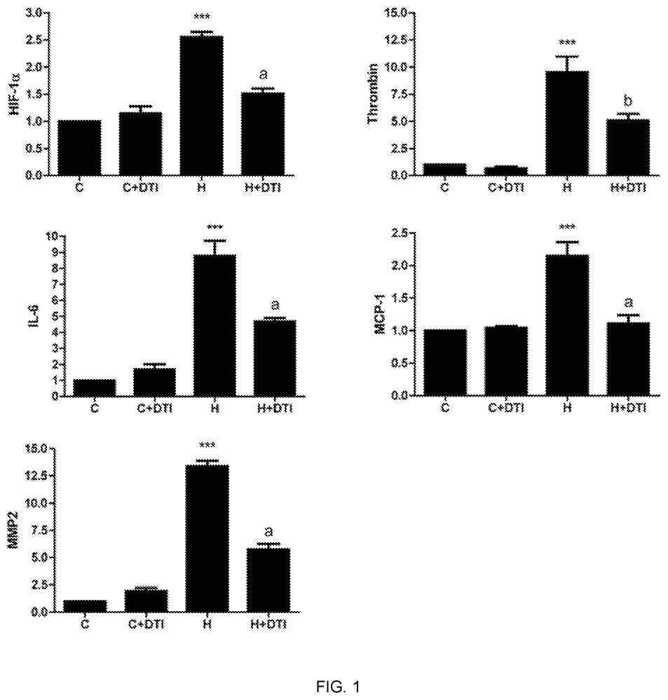

In contrast to the modest effects on cell survival, thrombin inhibition had profound effects on inflammatory gene expression in endothelial cell cultures exposed to hypoxia. Real-time PCR analysis of RNA collected from brain endothelial cells exposed to 6 h hypoxia showed a significant (p<0.001) increase in mRNA for HIF-1a as well as for several inflammatory-associated genes including thrombin, IL-6, MCP-1, and MMP2. Treatment of endothelial cells with the thrombin inhibitor (1 nM) significantly (p<0.01-0.001) reduced hypoxia-mediated effects on inflammatory gene expression (FIG. 1).

### Experiment 2: Treatment of AD Mice with DTI Reduces Cerebrovascular Expression of HIF-1a and Inflammatory Proteins

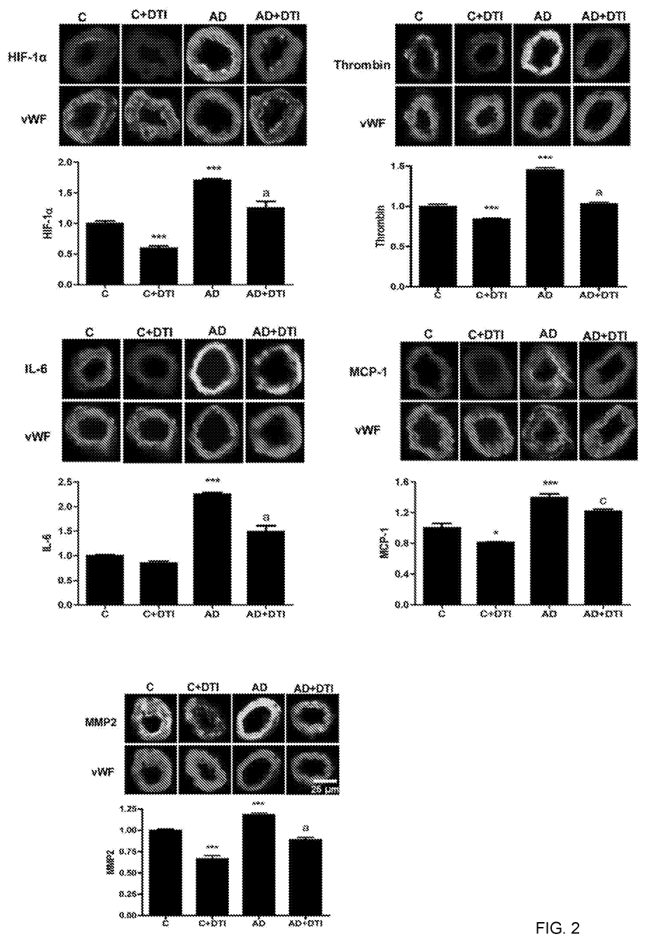

Micro-vessels from frontal cortex sections were examined by immunofluorescence from control and AD transgenic mice as well as control and AD mice that received 34 weeks of daily DTI administration (100 mg/kg). A comparison between control and AD mice showed that there was a significant (p<0.001) increase in cerebrovascular expression in AD mice of HIF-1 a, thrombin, IL-6, MCP-1, and MMP2 (FIG. 2). Treatment of AD mice with DTI significantly (p<0.050.001) reduced expression of these proteins (FIG. 2).

### Experiment 3: Dabigatran Reduces Expression of H/F-1a, Thrombin, IL-6, MCP-1, and MMP2 in the Brains of AD Transgenic Mice

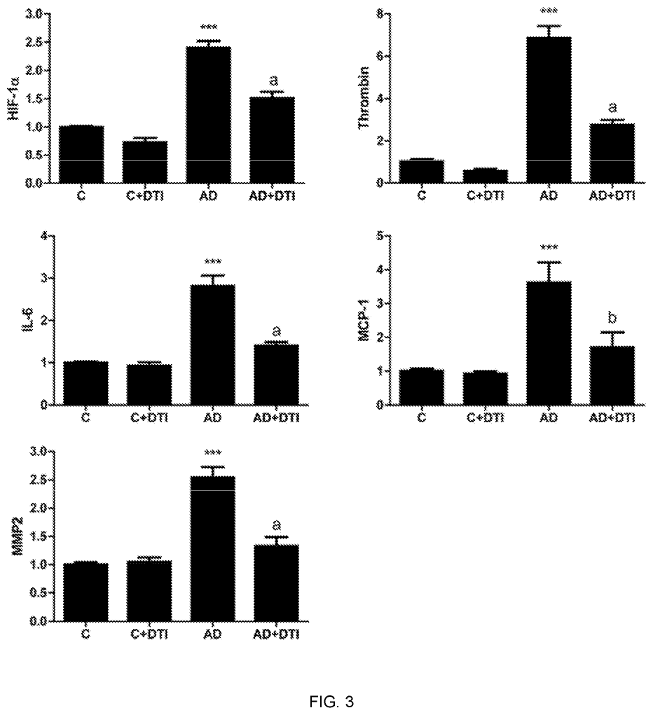

Examination of brain samples from AD transgenic mice by real time PCR demonstrated that expression of RNA for HIF-1a, thrombin, IL-6, MCP-1, and MMP2 was significantly (p<0.001) increased compared to levels in control animals. Similar to the data obtained for cerebrovascular expression (FIG. 2), examination of brain tissues also showed a significant (p<0.01 to p<0.001) decrease in RNA levels of these same proteins in AD mice that received DTI (FIG. 3).

### Experiment 4: Generation of ROS in AD Mice and Hypoxic Endothelial Cell Cultures is Inhibited by Dabigatran

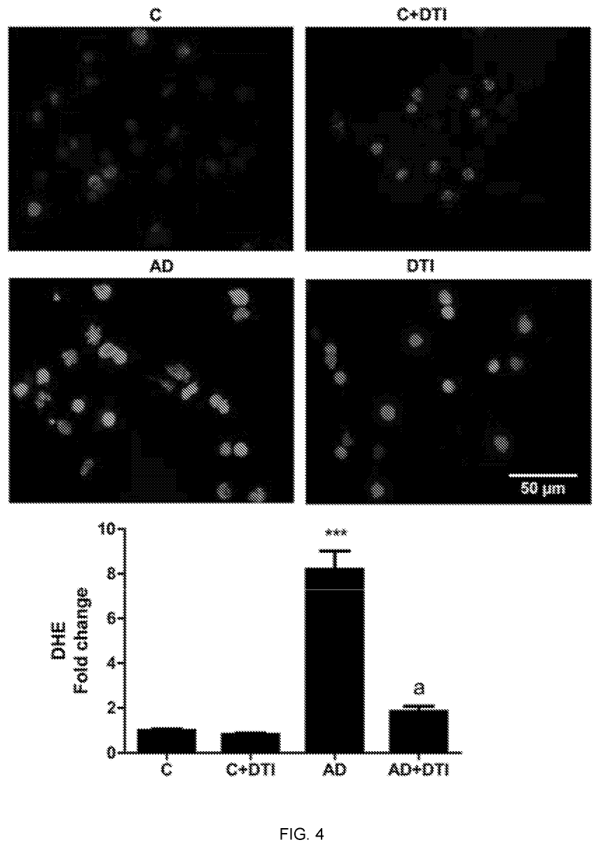

The effect of DTI on ROS level in brain tissue sections from frontal cortices of AD transgenic mice was assessed using the fluorescence dye DHE. Quantitation of DHE levels showed that ROS generation was 8-fold higher (p<0.001) in sections from AD mice brains compared to sections from control mice (FIG. 4). Administration of DTI for 34 weeks to AD mice significantly (p<0.001) decreased DHE level compared to untreated AD mice (FIG. 4).

An examination of cultured brain endothelial cells exposed to 6 h hypoxia showed a significant (p<0.001) increase in ROS levels, as assessed by an increase in DHE fluorescence. Treatment of endothelial cell cultures with DTI blocked (p<0.001) this increase in hypoxia-induced ROS generation:

### ROS Levels in Cultured Brain Endothelial Cells

| Control | Control + DTI | Hypoxia | Hypoxia + DTI |
|---|---|---|---|
| 1.00 ± 0.08 | 0.65± | 1.51 ± 0.19*** | 0.92 ± 0.06a |

Brain endothelial cell cultures were incubated in serum-free media or serum free media plus DTI (1 nM) and exposed to hypoxia (1% 02) or normoxic (21% 02) conditions for 6 h. The cells were incubated for 30 min with 5 pM of Dihydroethidium (DHE, red) fluorescent dye and NucBlue stain (blue). ROS generation was assessed by quantitation of signal intensities of DHE stained cells to non-stained cells. Data represent mean signal intensity of DHE stained cells to non-stained cells±SD from at least 3 separate experiments performed in duplicate. ***p<0.001 vs. Control; a p<0.001 vs. Hypoxia.

### Experiment 5: Treatment of Mice with Dabigatran

Fifteen-month-old Tg4510 mice (The Jackson Laboratory, Bar Harbor, Me.) treated for one week with vehicle dimethyl sulfoxide (DMSO) (Spectrum Chemicals & Laboratory Products, New Brunswick, N.J.) or Dabigatran (Pradaxa®, Boehringer Ingelheim Pharmaceuticals, Inc., Ridgefield, Conn.) at doses of 100 mg/kg via oral gavage. Wild-type control mice were treated with vehicle for one week. Brains were collected and homogenized and protein expression was evaluated using western blot.

### Current Clinical Trials

Phase I trials designed to evaluate thrombin inhibitors as potential contributors to the dementia and AD therapy pharmacopeia are currently in preparation. The current pilot protocol involves a randomized, double-blind placebo-controlled trail with dabigatran etexilate (Pradaxa®, Boehringer Ingelheim) and will be sponsored by the Alzheimer's Drug Discovery Foundation (ADDF).

We propose a 12-month treatment, with 3-month follow-up, parallel-arm, randomized, double-blind, placebo-controlled trial among patients with biomarker confirmed prodromal AD and mild AD cases (Clinical Dementia Rating global score of 0.5-1.0; n=15 dabigatran, n=15 placebo).

Aim 1: Determine the safety and tolerability of dabigatran treatment in AD patients 50 years of age.
Aim 2: Evaluate the effects of dabigatran on CSF and serum levels of IL-6 and TNFa.

Selection of subjects: Based on work in transgenic mice, we predict that subjects with early-stage AD are most likely to demonstrate changes in the key biomarker endpoints. To achieve the goals of this project, we will recruit subjects with MCI and early AD from the Butler Hospital Memory and Aging Program and The Alzheimer's Research Program at Rhode Island Hospital in Providence, R.I. These are two highly productive and experienced AD clinical trials programs affiliated with the Warren Alpert Medical School at Brown University. The George and Anne Ryan Institute for Neuroscience at the University of Rhode Island participates in a memorandum of understanding to facilitate collaborative translational research projects in neurodegenerative disorders among the University of Rhode Island, Brown University and the Brown Institute for Brain Sciences, Providence Va. Medical Center, Lifespan Health System and CARE New England.

Subjects will be randomized to a single starting dose (100 mg per day) of dabigatran or matching placebo over 12 months with a 3-month post-treatment follow-up. Dose of treatment and placebo (to maintain blind) will be tapered by medical monitor as needed. Subjects will be monitored for adverse events (AEs) and severe adverse events (SAEs) throughout the study. The 12-month course will provide an appropriate assessment of long-term safety and tolerability of a treatment that would require chronic use in this population. Further, it will improve the power to observe an impact of active treatment on neuroinflammatory markers that are expected to worsen, if modestly, over 12 months. The follow-up visit will be conducted at 3 months post-treatment, primarily to allow adequate drug wash-out and re-assess safety. All research procedures will be reviewed and approved by the Hospital Institutional Review Board prior to the initiation of the study.

Each patient will undergo an interview to include CDR administration and medical history and physical exam, as well as the following within 28 days prior to start of study drug: standard clinical labs, urine analysis, 12-lead echocardiogram (ECG) to establish left ventricular ejection fraction. If these results are within inclusion parameters, the subject will have imaging of the brain (MRI) if not available within previous 6 months, APOE genotype, serum biomarkers, lumbar puncture (LP) and CSF sent for biomarker analysis, and pregnancy test, if indicated, within 7 days prior to first study drug dose. Each patient will undergo MMSE, the Alzheimer's Disease Assessment Scale-Cognitive (ADAS-Cog) subscale and the CDR scale. Next patients will be randomized into one of the 2 arms of the study. At screening, patients will indicate their primary care providers, and, with patient's consent, a letter explaining study procedures will be sent to the provider. If any clinically important findings are noted during the course of the trial, these will be communicated to patients and families, and primary care providers. When a subject is eligible for the study, the coordinator will enter baseline and stratification data (MMSE Total Score, age and site) and will use the randomization protocol to assign the subject to one of the study arms.

Protocol: All participants will be evaluated monthly with clinical labs to monitor for AEs by the study coordinator and study physician to monitor for AEs outside of clinical labs. Only subjects who do not develop >grade 3 severe adverse reactions will be allowed to continue in the study (graded according to the National Cancer Institute Common Terminology Criteria for Adverse Events (CTCAE), version 3.0a). Neurological and physical exams will be conducted at screening and throughout study. Neuropsychological testing will be conducted at baseline, 6-month visit, 12-month visit, and 3-month follow-up post-therapy. Serum biomarkers and CSF assessment will be conducted at baseline, 12 month and 3-month follow-up post-therapy.

CSF and serum specimens will be processed at the clinic site. Samples will be transported to Dr. Grammas' laboratory at the University of Rhode Island for measurements of IL-6, TNFa and CSF AR using standard assays.

What is claimed is:

1. A method for treating a neurodegenerative disease in a patient in need thereof, comprising administering a therapeutic agent which inhibits vascular activation.

2. The method of claim 1, wherein said therapeutic agent is a direct thrombin inhibitor.

3. The method of claim 2, wherein the direct thrombin inhibitor is dabigatran.

4. The method of claim 1, wherein a level of at least one inflammatory protein in said patient's hippocampus is reduced, wherein the inflammatory proteins are chosen from the group consisting of TNFα, IL-6, IL-8, or MMP2.

5. The method of claim 1, wherein the level of oxidative stress in said patient's brain is reduced.

6. The method of claim 1, wherein said therapeutic is administered orally, parenterally, by inhalation spray, topically, rectally, nasally, buccally, vaginally or via an implanted reservoir.

7. The method of claim 1, wherein said neurodegenerative disease to be treated is selected from the group consisting of Alzheimer's disease, mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, Parkinson's disease, Parkinson's disease chemically induced by exposure to environmental agents, SNCA gene-linked Parkinson's disease, sporadic or idiopathic Parkinson's disease, or Parkin- or LRRK2-linked Parkinson's disease, autosomal-dominant Parkinson's disease, Diffuse Lewy Body Disease, Dementia with Lewy Bodies, Pure Autonomic Failure, Lewy body dysphagia, Incidental Dementia with Lewy Bodies, Inherited Dementia with Lewy Bodies, Multiple System Atrophy, Olivopontocerebellar Atrophy, Striatonigral Degeneration, Shy-Drager Syndrome, combined Alzheimer's and Parkinson disease and/or Multiple System Atrophy, Huntington's disease, synucleinopathies, disorders or conditions characterized by the presence of Lewy bodies, multiple sclerosis, Amyotrophic lateral sclerosis dementia, vascular dementia, Lewy body dementia, Parkinson's dementia, frontotemporal dementia, Down syndrome, psychosis, dyskinesia, agitation, conditions associated with dopaminergic therapy, synucleinopathies, diseases, disorders or conditions associated with abnormal expression, stability, activities and/or cellular processing of α-synuclein, disorders or conditions characterized by the presence of Lewy bodies, cognitive impairment and any combinations thereof.

8. The method of claim 7, wherein said neurodegenerative disease is Parkinson's disease chemically induced by exposure to environmental agents selected from the group consisting of pesticides, insecticides, herbicides, metals and any combination thereof.

9. The method of claim 8, wherein said metals are selected from the group consisting of manganese, aluminum, cadmium, copper, zinc or any combination thereof.

10. The method of claim 7, wherein said neurodegenerative disease is Inherited Dementia with Lewy Bodies and said Inherited Dementia with Lewy Bodies is caused by one or more mutations of the alpha-synuclein gene, PARK3 and PARK4.

11. The method of claim 7, wherein said psychosis is caused by caused by a neurodegenerative disease or associated with dopaminergic therapy.

12. The method of claim 11, wherein said dopaminergic therapy is used on a patient in need of such therapy to treat Parkinson's disease psychosis, Alzheimer's disease psychosis and/or Lewy body dementia psychosis.

13. The method of claim 7, wherein said dyskinesia is the result of agitation caused by a neurodegenerative disease or associated with dopaminergic therapy.

14. The method of claim 13, wherein said dopaminergic therapy is used on a patient in need of such therapy to treat Parkinson's disease psychosis, Alzheimer's disease psychosis and/or Lewy body dementia psychosis.

15. The method of claim 7, wherein said agitation is caused by a neurodegenerative disease or associated with dopaminergic therapy.

16. The method of claim 15, wherein said dopaminergic therapy is used on a patient in need of such therapy to treat Parkinson's disease psychosis, Alzheimer's disease psychosis and/or Lewy body dementia psychosis.

17. The method of claim 7, wherein said condition associated with dopaminergic therapy is selected from the group consisting of dystonia, myoclonus and tremors.
